(19) United States
(12) Patent Application Publication
(10) Pub. No.: US 2004/0002545 A1
(43) Pub. Date: Jan. 1, 2004

(54) Thiazole derivatives of 2-methoxyimino-2-(pyridinyloxymethyl)-phenyl-acetamides useful as fungicides

(76) Inventors: Jeannie Rachel Cetusic, Avon, IN (US); Brent Jeffrey Rieder, Greenfield, IN (US)

Correspondence Address: DOW AGROSCIENCES LLC, 9330 ZIONSVILLE RD, INDIANAPOLIS, IN, 46268, US

(21) Appl. No.: 10/362,812

(22) PCT Filed: Sep. 19, 2001
(86) PCT No.: PCT/US01/29351

(51) Int. Cl. A61K 31/15

(52) U.S. Cl. 514/640

(57) ABSTRACT

The present invention provides 2-methoxyimino-2(pyridinyloxymethyl) phenyl acetamides with a isothiazolyl ring on the pyridine ring according to formula (I) as well as their use as fungicidal compounds.

### PRIORITY

This application claims priority from U.S. provisional application 60/233,781 which was filed on Sep. 19, 2001.

### FIELD OF THE INVENTION

This invention is related to the field of compounds having fungicidal activity and processes to make and use such compounds.

### BACKGROUND OF THE INVENTION

Our history is riddled with outbreaks of fungal diseases that have caused widespread human suffering. One need look no further than the Irish potato famine of the 1850's, where an estimated 1,000,000 people died, to see the effects of a fungal disease.

Fungicides are compounds, of natural or synthetic origin, which act to protect plants against damage caused by fungi. Current methods of agriculture rely heavily on the use of fungicides. In fact, some crops cannot be grown usefully without the use of fungicides.

Using fungicides allows a grower to increase the yield of the crop and consequently, increase the value of the crop. In most situations, the increase in value of the crop is worth at least three times the cost of the use of the fungicide. However, no one fungicide is useful in all situations.

Consequently, research is being conducted to produce fungicides that are safer, that have better performance, that are easier to use, and that cost less. In light of the above, the inventors provide this invention.

### SUMMARY OF THE INVENTION

It is an object of this invention to provide compounds that have fungicidal activity. It is an object of this invention to provide processes that produce compounds that have fungicidal activity.

It is an object of this invention to provide processes that use compounds that have fungicidal activity.

In accordance with this invention, processes to make and processes to use compounds having a general formula according to formula one, and said compounds are provided.

While all the compounds of this invention have fungicidal activity, certain classes of compounds may be preferred for reasons such as, for example, greater efficacy or ease of synthesis.

### DETAILED DESCRIPTION OF THE INVENTION

The compounds have a formula according to formula one. In formula one:

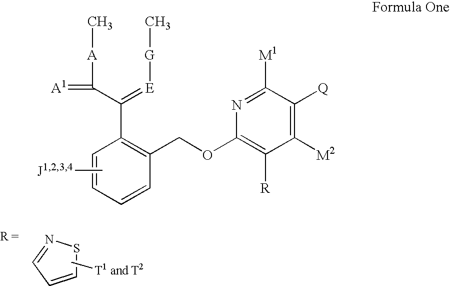

A is selected from the group consisting of oxy (—O—) and amino (—NH—);

A¹ is selected from the group consisting of oxo (O═) and thioxo (S═);

E is selected from the group consisting of aza (—N═) and methine (—CH═);

J¹, J², J³, and J⁴ are independently selected from the group consisting of hydro (—H), halo (—F, —Cl, —Br, and —I), C₁₋₄ alkyl, C₁₋₄ alkoxy, C₁₋₄ alkyl (mono or multi-halo), and C₁₋₄ alkylthio;

M¹, and M² are selected from the group consisting of hydro (—H), halo (—F, —Cl, —Br, and —I), C₁₋₆ alkyl, C₁₋₆ alkoxy, C₁₋₄ alkyl (mono or multi-halo), and C₁₋₄ alkylthio, nitro (—NO₂), (mono or multi-halo) C₁₋₄ alkoxy, aryl (-Aryl), substituted aryl (—SAryl), heteroaryl (—HAryl), and substituted heteroaryl (—SHAryl), where “aryl” or “Ph” refers to a phenyl group and where “heteroaryl” refers to pyridyl, pyridinyl, pyrazinyl or pyridazinyl, and where said SAryl and SHAryl have substituents that are independently selected from the group consisting C₁-C₆ alkyl, C₁-C₆ alkoxy, halo-C₁-C₆ alkyl, halo-C₁-C₆ alkoxy, halo, nitro, carbo-C₁-C₆ alkoxy, or cyano, arylalkyl, alkanoyl, benzoyl, amino, and substituted amino, preferably, hydro (—H), C₁-C₆ alkyls, arylalkyl, alkanoyl, benzoyl, amino, and substituted amino where said substituted amino has substituents that are independently selected from the group consisting of hydro (—H), alkyl, arylalkyl, alkanoyl, benzoyl, and amino;

Q is selected from the group consisting of hydro, halo, cyano, (mono or multi halo) C₁₋₆ alkyl, and C₁₋₆ alkyl; and

T¹ and T² are independently selected from the group consisting of hydro (—H), halo (—F, —Cl, —Br, and —I), C₁₋₆ alkyl, C₁₋₆ alkoxy, C₁₋₄ alkyl (mono or multi-halo), and C₁₋₄ alkylthio, nitro (—NO₂), (mono or multi-halo) C₁₋₄ alkoxy, aryl (-Aryl), substituted aryl (—SAryl), heteroaryl (—HAryl), and substituted heteroaryl (—SHAryl), where “aryl” or “Ph” refers to a phenyl group and where “heteroaryl” refers to pyridyl, pyridinyl, pyrazinyl or pyridazinyl, and where said SAaryl and SHAryl have substituents that are independently selected from the group consisting C₁-C₆ alkyl, C₁-C₆ alkoxy, halo-C₁-C₆ alky, halo-C₁-C₆ alkoxy, halo, nitro, carbo-C₁-C₆ alkoxy, or cyano, arylalkyl, alkanoyl, benzoyl, amino, and substituted amino, preferably, hydro (—H), C₁-C₆ alkyls, arylalkyl, alkanoyl, benzoyl, amino, and substituted amino where said substituted amino has substituents that are independently selected from the group consisting of hydro (—H), alkyl, arylalkyl, alkanoyl, benzoyl, and amino. C₁₋₄ alkyl or one of the single bonds can be the connecting bond to the pyridyl.

The term “alkyl”, “alkenyl”, or “alkynyl” refers to an unbranched or branched chain carbon group. The term “alkoxy” refers to an unbranched or branched chain alkoxy group. The term “haloalkyl” refers to an unbranched or branched alkyl group substituted with one or more halo atoms. The term “haloalkoxy” refers to an alkoxy group substituted with one or more halo atoms. Throughout this document, all temperatures are given in degrees Celsius and all percentages are weight percentages, unless otherwise stated. The term “Me” refers to a methyl group. The term “Et” refers to an ethyl group. The term “Pr” refers to a propyl group. The term “Bu” refers to a butyl group. The term “EtOAc” refers to ethyl acetate. The term “DMSO” refers to dimethylsulfoxide. The ten “Ether”, when used in the body of text under “Preparation”, refers to diethyl ether. The term “ppm” refers to parts per million. The term, “psi” refers to pounds per square inch.

In general, these compounds can be used in a variety of ways. These compounds are preferably applied in the form of a formulation comprising one or more of the compounds with a phytologically acceptable carrier. Concentrated formulations can be dispersed in water, or another liquid, for application, or formulations can be dust-like or granular, which can then be applied without further treatment. The formulations are prepared according to procedures which are conventional in the agricultural chemical art, but which are novel and important because of the presence therein of one or more of the compounds.

The formulations that are applied most often are aqueous suspensions or emulsions. Either such water-soluble, water suspendable, or emulsifiable formulations are solids, usually known as wettable powders, or liquids, usually known as emulsifiable concentrates, aqueous suspensions, or suspension concentrates. The present invention contemplates all vehicles by which one or more of the compounds can be formulated for delivery and use as a fungicide.

As will be readily appreciated, any material to which these compounds can be added may be used, provided they yield the desired utility without significant interference with the activity of these compounds as antifungal agents.

Wettable powders, which may be compacted to form water dispersible granules, comprise an intimate mixture of one or more of the compounds, an inert carrier and surfactants. The concentration of the compound in the wettable powder is usually from about 10% to about 90% w/w, more preferably about 25% to about 75% w/w. In the preparation of wettable powder formulations, the compounds can be compounded with any of the finely divided solids, such as prophyllite, talc, chalk, gypsum, Fuller's earth, bentonite, attapulgte, starch, casein, gluten, montmorillonite clays, diatomaceous earths, purified silicates or the like. In such operations, the finely divided carrier is ground or mixed with the compounds in a volatile organic solvent. Effective surfactants, comprising from about 0.5% to about 10% of the wettable powder, include sulfonated lignins, naphthalenesulfonates, alkylbenzenesulfonates, alkyl sulfates, and non-ionic surfactants, such as ethylene oxide adducts of alkyl phenols.

Emulsifiable concentrates of the compounds comprise a convenient concentration, such as from about 10% to about 50% w/w, in a suitable liquid. The compounds are dissolved in an inert carrier, which is either a water miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, such as, for example, terpenic solvents, including rosin derivatives, aliphatic ketones, such as cyclohexanone, and complex alcohols, such as 2-ethoxyethanol.

Emulsifiers which can be advantageously employed herein can be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulphonic acids, oil soluble salts or sulphated polyglycol ethers and appropriate salts of phosphated polyglycol ether.

Representative organic liquids which can be employed in preparing the emulsifiable concentrates of the present invention are the aromatic liquids such as xylene, propyl benzene fractions; or mixed naphthalene fractions, mineral oils, substituted aromatic organic liquids such as dioctyl phthalate; kerosene; dialkyl amides of various fatty acids, particularly the dimethyl amides of fatty glycols and glycol derivatives such as the n-butyl ether, ethyl ether or methyl ether of diethylene glycol, and the methyl ether of triethylene glycol. Mixtures of two or more organic liquids are also often suitably employed in the preparation of the emulsifiable concentrate. The preferred organic liquids are xylene, and propyl benzene fractions, with xylene being most preferred. The surface-active dispersing agents are usually employed in liquid formulations and in the amount of from 0.1 to 20 percent by weight of the combined weight of the dispersing agent with one or more of the compounds. The formulations can also contain other compatible additives, for example, plant growth regulators and other biologically active compounds used in agriculture.

Aqueous suspensions comprise suspensions of one or more water-insoluble compounds, dispersed in an aqueous vehicle at a concentration in the range from about 5% to about 50% w/w. Suspensions are prepared by finely grinding one or more of the compounds, and vigorously mixing the ground material into a vehicle comprised of water and surfactants chosen from the same types discussed above. Other ingredients, such as inorganic salts and synthetic or natural gums, may also be added to increase the density and viscosity of the aqueous vehicle. It is often most effective to grind and mix at the same time by preparing the aqueous mixture and homogenizing it in an implement such as a sand mill, ball mill, or piston-type homogenizer.

The compounds may also be applied as granular formulations, which are particularly useful for applications to the soil. Granular formulations usually contain from about 0.5% to about 10% w/w of the compounds, dispersed in an inert carrier which clay or a similar inexpensive substance. Such formulations are usually prepared by dissolving the compounds in a suitable solvent and applying it to a granular carrier which has been preformed to the appropriate particle size, in the range of from about 0.5 to about 3 mm. Such formulations may also be prepared by making a dough or paste of the carrier and the compound, and crushing and drying to obtain the desired granular particle.

Dusts containing the compounds are prepared simply by intimately mixing one or more of the compounds in powdered form with a suitable dusty agricultural carrier, such as, for example, kaolin clay, ground volcanic rock, and the like. Dusts can suitably contain from about 1% to about 10% w/w of the compounds.

The formulations may contain adjuvant surfactants to enhance deposition, wetting and penetration of the compounds onto the target crop and organism. These adjuvant surfactants may optionally be employed as a component of the formulation or as a tank mix. The amount of adjuvant surfactant will vary from 0.01 percent to 1.0 percent v/v based on a spray-volume of water, preferably 0.05 to 0.5%. Suitable adjuvant surfactants include ethoxylated nonyl phenols, ethoxylated synthetic or natural alcohols, salts of the esters or sulphosuccinic acids, ethoxylated organosilicones, ethoxylated fatty amines and blends of surfactants with mineral or vegetable oils.

The formulations may optionally include combinations that can comprise at least 1% of one or more of the compounds with another pesticidal compound. Such additional pesticidal compounds may be fungicides, insecticides, nematocides, miticides, arthropodicides, bactericides or combinations thereof that are compatible with the compounds of the present invention in the medium selected for application, and not antagonistic to the activity of the present compounds. Accordingly, in such embodiments the other pesticidal compound is employed as a supplemental toxicant for the same or for a different pesticidal use. The compounds and the pesticidal compound in the combination can generally be present in a weight ratio of from 1:00 to 100:1.

The present invention includes within its scope methods for the control or prevention of fungal attack. These methods comprise applying to the locus of the fungus, or to a locus in which the infestation is to be prevented (for example applying to cereal or grape plants), a fungicidal amount of one or more of the compounds. The compounds are suitable for treatment of various plants at fungicidal levels, while exhibiting low phytotoxicity. The compounds are useful in a protectant or eradicant fashion. The compounds are applied by any of a variety of known techniques, either as the compounds or as formulations comprising the compounds. For example, the compounds may be applied to the roots, seeds or foliage of plants for the control of various fungi, without damaging the commercial value of the plants. The materials are applied in the form of any of the generally used formulation types, for example, as solutions, dusts, wettable powders, flowable concentrates, or emulsifiable concentrates. These materials are conveniently applied in various known fashions.

The compounds have been found to have significant fungicidal effect particularly for agricultural use. Many of the compounds are particularly effective for use with agricultural crops and horticultural plants, or with wood, paint, leather or carpet backing.

In particular, the compounds effectively control a variety of undesirable fungi that infect useful plant crops. Activity has been demonstrated for a variety of fungi, including for example the following representative fungi species:

Downy Mildew of Grape (Plasmopara viticola—PLASVI);

Late Blight of Tomato (Phytophthora infestans—PHYTIN);

Apple Scab (Venturia inaequalis—VENTIN);

Brown Rust of Wheat (Puccinia recondita—PUCCRT);

Stripe Rust of Wheat (Puccinia striiformis—PUCCST);

Rice Blast (Pyricularia oryzae—PYRIOR);

Cercospora Leaf Spot of Beet (Cercospora beticola—CERCBE);

Powdery Mildew of Wheat (Erysiphe graminis—ERYSGT);

Leaf Blotch of Wheat (Septoria tritici—SEPTTR);

Sheath Blight of Rice (Rhizoctonia solani—RHIZSO);

Eyespot of Wheat (Pseudocercosporella herpotrichoides—PSDCHE);

Brown Rot of Peach (Monilinia fructicola—MONIFC); and

Glume Blotch of Wheat (Septoria nodorum—LEPTNO).

It will be understood by those in the art that the efficacy of the compound for the foregoing fungi establishes the general utility of the compounds as fungicides. The compounds have broad ranges of efficacy as fungicides. The exact amount of the active material to be applied is dependent not only on the specific active material being applied, but also on the particular action desired, the fungal species to be controlled, and the stage of growth thereof, as well as the part of the plant or other product to be contacted with the compound. Thus, all the compounds, and formulations containing the same, may not be equally effective at similar concentrations or against the same fungal species.

The compounds are effective in use with plants in a disease inhibiting and phytologically acceptable amount. The term “disease inhibiting and phytologically acceptable amount” refers to an amount of a compound that kills or inhibits the plant disease for which control is desired, but is not significantly toxic to the plant. This amount will generally be from about 1 to about 1000 ppm, with 10 to 500 ppm being preferred. The exact concentration of compound required varies with the fungal disease to be controlled, the type of formulation employed, the method of application, the particular plant species, climate conditions, and the like. A suitable application rate is typically in the range from about 0.10 to about 4 pounds/acre.

### EXAMPLES

These examples are provided to further illustrate the invention. They are not meant to be construed as limiting the invention.

Preparation of 5-bromo-3-methylisothiazole (Compound A) and 4,5-dibromo-3-methyliso-thiazole (Compound B)

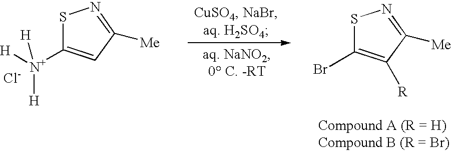

5-Amino-3-methylisothiazole hydrochloride (1.0 g; 6.7 mmol) was dissolved in 9M sulfuric acid (13.4 mL) at RT. Copper(II)sulfate (2.7 g; 16.8 mmol; 2.5 eq) and sodium bromide (2.4 g; 23.5 mmol; 3.5 eq) were added, and the resulting thick mixture was cooled to 0° C. in an ice-salt bath. A solution of sodium nitrite (5.8 mg; 7.4 mmol; 1.1 eq) in water (2.5 mL) was added slowly dropwise keeping the internal temperature <10° C. When the addition was complete, stirring continued at 0° C. for 20 minutes and then at RT for 30 minutes until nitrogen evolution was no longer visible. The reaction mixture was poured into water (exotherm) where it stirred until most of the solids dissolved. It was transferred to a separatory funnel and extracted with diethyl ether (×3). The combined organic layers were washed with brine and dried over sodium sulfate. After careful removal of solvent (no heat) the crude residue was purified by flash chromatography (5% ethyl acetate/hexanes) to give Compound A (confirmed by GCMS, m/e 179) in 28% yield (330 mg) and Compound B (m/e 257) in a smaller amount (yield not measured).

Preparation of 4,5-dibromo-3-methyliso-thiazole (Compound B) Method Two.

5-Amino-3-methylisothiazole hydrochloride (3.1 g; 20 mmol) was equilibrated between ethyl acetate and 10% sodium carbonate. The organic layer was filtered and evaporated in vacuo to 2.25 g (˜20 mmol) of 5-amino-3-methylisothiazole. It was pulverized and added to 100 mL 48% hydrobromic acid. 1.5 g (22 mmol) sodium nitrite was dissolved in 5 mL water and added to the starting material solution at room temperature. When the resultant exotherm was complete, 5.8 g (40 mmol) of pulverized cuprous bromide was added with stirring and left at room temperature ˜5 hours. The mixture was flooded with 200 mL water, then extracted with 1:1 ether/pentane. The organic layer was filtered and evaporated in vacuo to ˜2 g of a yellow gum. Thin layer chromatography (SiO₂/ether/hexane) showed a small fast spot and a large, slightly slower spot. Dissolution in pentane with a minimum volume of ether, followed by an extractive wash with conc. hydrochloric acid removed all of the faster spot. Subsequent neutralization of the latter with 10% ammonium hydroxide and ether extraction, followed by evaporation of the extract, yielded 100 mg of Compound A with correct spectral data. The acid-washed ether/pentane layer was filtered and evaporated in vacuo to 1 g of the major spot, a low-melting orange solid. It was confirmed to be Compound B by GCMS (m/e 257) and 1H NMR (singlet at 2.6 ppm), in 39% recovered yield.

Preparation of 3-chloro-2-fluoro-(3-methyl-5-isothiazolyl)pyridine (Compound C)

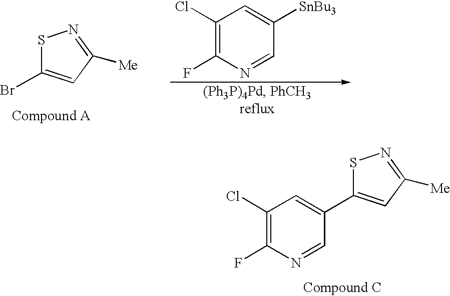

5-Bromo-3-methylisothiazole (400 mg; 2.2 mmol; 1.1 eq) (Compound A) was dissolved in toluene (5 mL) and tetrakis(triphenylphosphine)palladium(0) (116 mg; 0.1 mmol; 0.05 eq) was added. This mixture was blanketed with nitrogen and heated to 90° C. for the addition of a solution of 3-chloro-2-fluoro-5-(tributylstannyl)pyridine (838 mg; 2.0 mmol; 1 eq) in toluene (2 mL). This mixture was then heated to reflux overnight. It was cooled to RT, diluted with ether and filtered through Celite to give an orange solution which became a yellow-orange solid when the solvent was removed. GCMS showed 2 major products corresponding to the desired Compound C (m/e 228).

Preparation of 2-fluoro-3-methyl-(4bromo-3-methyl-5-isothiazolyl)pyridine (Compound D)

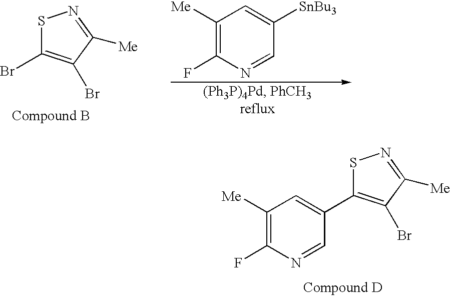

4,5-Dibromo-3-methylisothiazole (950 mg; 4 mmol) (Compound B) was dissolved in toluene (75 mL) and tetrakis(triphenylphosphine)palladium(0) (240 mg; 0.2 mmol) was added. This mixture was blanketed with nitrogen and heated to 90° C. for the addition of a solution of 2-fluoro-3-methyl-5-(tributylstannyl)pyridine (1.6 g; 4.0 mmol; 1 eq) in toluene (2 mL). This mixture was then heated to reflux 2.5 hrs. Thin layer chromatography (hexane/ether) showed no Compound B, and the presence of a large mid-Rf product spot. The suspension was filtered and the filtrate stored cold overnight. After evaporating in vacuo to a dark oil it was eluted on a silica column with 1:1 pentane/ether to collect 0.8 g of the major, desired product as a clear oil. GCMS m/e=286/288 (confirming), 70% yield.

Preparation of 2-[[[3-chloro-5-[5-[3-methylisothiazoly]]-2-pyridinyl]-oxy]methyl]-alpha-(methoxyimino)-N-methylbenzeneacetamide (Compound 1).

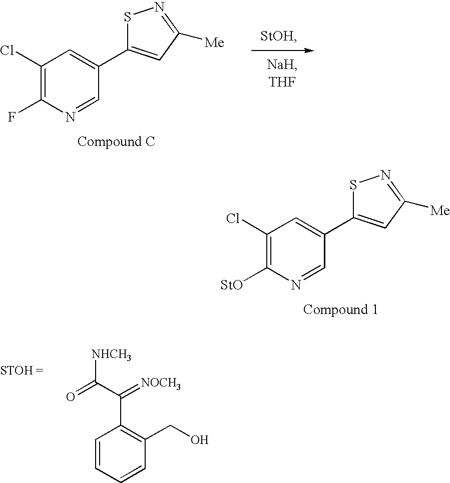

StOH (0.33 g, 0.0015 mol) was dissolved with stirring in dry THF (10 mL) and 60% sodium hydride (0.07 g, 0.0018 mol) added. The mixture was stirred at room temperature for 30 minutes and a solution of compound C (0.29 g, 0.0014 mol) in dry THF (5 mL) added. The mixture was heated with stirring at 50° C. for 5 hours, cooled, and poured into water. The mixture was extracted with ethyl acetate (40 mL) and the organic extracts washed with water (40 mL) and brine (40 mL), and dried over anhydrous sodium sulphate. Evaporation of the solvent under reduced pressure and purification of the residue by chromatography over silica (10-50% ethyl acetate/hexanes) gave the desired product.

Preparation of 2-[[[3-methyl-5-[5-[4-bromo-3-methylisothiazolyl]]-2-pyridinyl]-oxy]methyl]-alpha-(methoxyimino)-N-methylbenzeneacetamide (Compound 2).

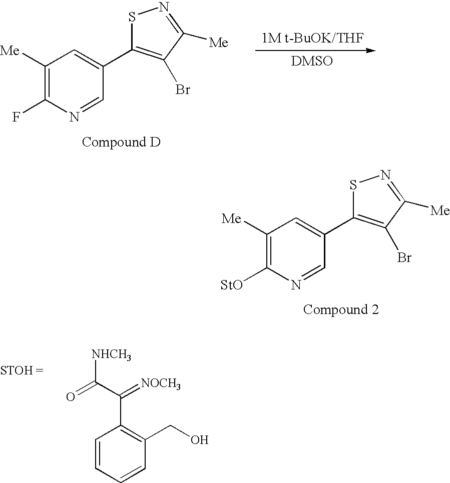

Compound D (0.72 g, 2.5 mmol) was dissolved in 50 mL anh. DMSO. To this was added 0.56 g (2.5 mmol) of the methoximinoamide referred to as StOH, with stirring and nitrogen purging. Upon injection of 3 mL (3 mmol) of 1M t-BuOK/THF the solution turned deep red. After stirring 20 min. thin layer chromatography of an acidified aliquot showed no Compound D, and a large low-mid-Rf product spot. Removed most of the DMSO in vacuo, flooded with 100 mL dilute hydrochloric acid (pH 4-5), and extracted twice with ethyl acetate. Filtered and evaporated extract in vacuo to 1.1 g orange gum. Eluted on silica column with 5:2 ether/pentane to collect 0.75 g of the major product as a clear oil which became a hard white foam on extended high vacuum, mp=48-53° C. GCMS m/e=490 (confirming).

### Biological Results

Pathogen propagation and host inoculation. Plants were inoculated with various pathogens 1-4 days before compound application (curative tests) and 1-7 days after compound application (protectant tests). For all wheat trials, compounds were applied at growth stage 1.2, when the second leaf was expanded to about ½ of its final size (12 days after seeds were first watered). Information on the growth stages of other plant species at the time compound application and on the propagation and inoculation procedures associated with each pathogen is given below.

ERYSGT: Wheat seedlings were infected with fresh spores from the obligate pathogen ERYSGT by shaking heavily infected wheat plants over them. Plants that had been dusted with ERYSGT spores were incubated in the greenhouse at 22° C. until disease symptoms had fully developed (usually 7 days).

PUCCRT: Spores of the obligate pathogen PUCCRT were collected from infected plants with a vacuum apparatus and stored at 4° C. Approximately 0.1 g of fresh spores (stored at 4° C. for less than 30 days) was mixed with several drops of Tween 20. The thick spore paste was diluted to 100 ml with water and sprayed to run-off on wheat seed seedlings. Plants inoculated with PUCCRT were kept in a 20° C. dew chamber overnight and then transferred to a 20° C. growth chamber where symptoms developed in 8-9 days.

SEPTTR: Fresh inoculum is prepared in a manner similar to that described for LEPTNO. In this case, a brownish layer of spores covers the entire surface of the PDA plate and only a few plates are needed to obtain a large number of spores. After incubation overnight in the 20° C. dew chamber, inoculated plants were continually misted for 3 days in a 20° C. greenhouse, then grown at 20° C. without mist until disease symptoms had fully developed (usually about 10 days).

LEPTNO: Fresh inoculum was prepared by streaking PDA plates with spore exudates from an older plate using a sterile spatula. The plates were incubated at 18° C. under black lights and typically produced large quantities of spores in pink exudate in 6-7 days. A small amount of tap water was poured onto several plates and spores were collected by scraping the exudates off the PDA surface into the water. The spore solutions were combined in a large beaker, diluted with 200-300 ml of water and filtered through a 180 u mesh screen. The spore concentration was determined using a hemacytometer and water was added to obtain a final concentration of 10⁷ spores/ml. Approximately 3 large drops of Tween 20 were added for each 100 ml of volume and the spore solution was sprayed to run-off on wheat seedlings. Inoculated plants were placed in a 20° C. dew chamber overnight, then moved to a 20° C. greenhouse where they were continually misted (12 seconds of mist every minute) until disease symptoms were fully developed (8-10 days).

TABLE ONE
“Biological Data for Compounds 1-2

| | Rate | ERYSGT | ERYSGT | LEPTNO | LEPTNO | PUCCRT | PUCCRT | SEPTTR | SEPTTR |
|---|---|---|---|---|---|---|---|---|---|
| Compound | (g ai/ha) | 3DC | 7DP | 3DC | 7DP | 3DC | 7DP | 7DP | 3DC |
| 1 | 125 | 97 | 100 | 71 | 98 | 100 | 100 | 91 | 99 |
| | 62.5 | 94 | 88 | 53 | 95 | 100 | 100 | 76 | 85 |
| | 31.3 | 92 | 45 | 60 | 50 | 99 | 94 | 88 | 59 |
| | 15.5 | 88 | 51 | 58 | 46 | 93 | 80 | 53 | 22 |
| 2 | 125 | failed | failed | 67 | 98 | 93 | 100 | 97 | 95 |
| | 62.5 | | | 47 | 84 | 93 | 100 | 96 | 90 |
| | 31.3 | | | 73 | 79 | 63 | 100 | 85 | 83 |
| | 15.5 | | | 65 | 65 | 50 | 100 | 85 | 97 |

We claim:

1. A compound according to formula one 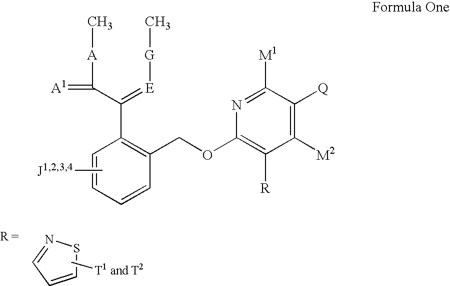
A is selected from the group consisting of oxy (—O—) and amino (—NH—);
A¹ is selected from the group consisting of oxo (O═) and thioxo (S═);
E is selected from the group consisting of aza (—N═) and methine (—CH═);
J¹, J², J³, and J⁴ are independently selected from the group consisting of hydro (—H), halo (—F, —Cl, —Br, and —I), C₁₋₄ alkyl, C₁₋₄ alkoxy, C₁₋₄ alkyl (mono or multi- halo), and C₁₋₄ alkylthio;
M¹, and M² are selected from the group consisting of hydro (—H), halo (—F, —Cl, —Br, and —I), C₁₋₆ alkyl, C₁₋₆ alkoxy, C₁₋₄ alkyl (mono or multi-halo), and C₁₋₄ alkylthio, nitro (—NO₂), (mono or multi-halo) C₁₋₄ alkoxy, aryl (-Aryl), substituted aryl (—SAryl), heteroaryl (—HAryl), and substituted heteroaryl (—SHAryl), where “aryl” or “Ph” refers to a phenyl group and where “heteroaryl” refers to pyridyl, pyridinyl, pyrazinyl or pyridazinyl, and where said SAryl and SHAryl have substituents that are independently selected from the group consisting C₁-C₆ alkyl, C₁-C₆ alkoxy, halo-C₁-C₆ alkyl, halo-C₁-C₆ alkoxy, halo, nitro, carbo-C₁-C₆ alkoxy, or cyano, arylalkyl, alkanoyl, benzoyl, amino, and substituted amino, preferably, hydro (—H), C₁-C₆ alkyls, arylalkyl, alkanoyl, benzoyl, amino, and substituted amino where said substituted amino has substituents that are independently selected from the group consisting of hydro (—H), alkyl, arylalkyl, alkanoyl, benzoyl, and amino;
Q is selected from the group consisting of hydro, halo, cyano, (mono or multi halo) C₁₋₆ alkyl, and C₁₋₆ alkyl; and
T¹ and T² are independently selected from the group consisting of hydro (—H), halo (—F, —Cl, —Br, and —I), C₁₋₆ alkyl, C₁₋₆ alkoxy, C₁₋₄ alkyl (mono or multi-halo), and C₁₋₄ alkylthio, nitro (—NO₂), (mono or multi-halo) C₁₋₄ alkoxy, aryl (-Aryl), substituted aryl (—SAryl), heteroaryl (—HAryl), and substituted heteroaryl (—SHAryl), where “aryl” or “Ph” refers to a phenyl group and where “heteroaryl” refers to pyridyl, pyridinyl, pyrazinyl or pyridazinyl, and where said SAaryl and SHAryl have substituents that are independently selected from the group consisting C₁-C₆ alkyl, C₁-C₆ alkoxy, halo-C₁-C₆ alkyl, halo-C₁-C₆ alkoxy, halo, nitro, carbo-C₁-C₆ alkoxy, or cyano, arylalkyl, alkanoyl, benzoyl, amino, and substituted amino, preferably, hydro (—H), C₁-C₆ alkyls, arylalkyl, alkanoyl, benzoyl, amino, and substituted amino where said substituted amino has substituents that are independently selected from the group consisting of hydro (—H), alkyl, arylalkyl, alkanoyl, benzoyl, and amino. C₁₋₄ alkyl or one of the single bonds can be the connecting bond to the pyridyl.

2. A compound according to claim 1 wherein A is amino (—NH—).

3. A compound according to claim 1 wherein A¹ is oxo (O═).

4. A compound according to claim 1 wherein E is aza (—N═).

5. A compound according to claim 1 wherein J¹, J², J³, and J⁴ are hydro (—H).

6. A compound according to claim 1 wherein M¹, and M² are hydro (—H).

7. A compound according to claim 1 wherein Q is halo.

8. A compound according to claim 1 wherein T¹ and T² are independently selected from the group consisting of hydro (—H), halo (—F, —Cl, —Br, and —I), and C₁₋₆ alkyl.

9. A compound according to claim 1 wherein A is amino (—NH—); A¹ is oxo (O═); E is aza (—N═); J¹, J², J³, and J⁴ are hydro (—H); M¹, and M² are hydro (—H); Q is halo; and T¹ and T² are independently selected from the group consisting of hydro (—H), halo (—F, —Cl, —Br, and —I), and C₁₋₆ alkyl

10. A process comprising applying fungicidal amount of a compound according to claim 1 to control or prevent a fungal attack in a locus.

11. A process comprising applying fungicidal amount of a compound according to claim 2 to control or prevent a fungal attack in a locus.

12. A process comprising applying fungicidal amount of a compound according to claim 3 to control or prevent a fungal attack in a locus.

13. A process comprising applying fungicidal amount of a compound according to claim 4 to control or prevent a fungal attack in a locus.

14. A process comprising applying fungicidal amount of a compound according to claim 5 to control or prevent a fungal attack in a locus.

15. A process comprising applying fungicidal amount of a compound according to claim 6 to control or prevent a fungal attack in a locus.

16. A process comprising applying fungicidal amount of a compound according to claim 7 to control or prevent a fungal attack in a locus.

17. A process comprising applying fungicidal amount of a compound according to claim 8 to control or prevent a fungal attack in a locus.

18. A process comprising applying fungicidal amount of a compound according to claim 9 to control or prevent a fungal attack in a locus.
